(19) United States
(12) Patent Application Publication
(10) Pub. No.: US 2026/0242771 A1
(43) Pub. Date: Aug. 20, 2026

(54) LECTIN-ASSISTED ISOLATION OF MAMMALIAN CELL NUCLEI

(71) Applicant: TALUS BIOSCIENCE, INC., Seattle, WA (US)

(72) Inventors: Julia E. ROBBINS, Seattle, WA (US); Daniele CANZANI, Seattle, WA (US); Erin F. BRODERICK, Seattle, WA (US); Noelle C. SCHUMACKER, Seattle, WA (US); Lindsay K. PINO, Seattle, WA (US); Alexander J. FEDERATION, Renton, WA (US)

(21) Appl. No.: 19/152,847

(22) PCT Filed: Feb. 2, 2024
(86) PCT No.: PCT/US2024/014339

§ 371 Date: Jul. 31, 2025

Related U.S. Application Data
(60) Provisional application No. 63/443,201, filed on Feb. 3, 2023.

(51) Int. Cl. C12N 15/10; C07K 14/47; C12M 1/00; C12M 1/12; C12M 1/32; C12M 1/42

(52) U.S. Cl. CPC C12N 15/1013; C07K 14/47; C12M 23/12; C12M 25/16; C12M 35/06; C12M 47/06

(57) ABSTRACT

The present disclosure provides methods and compositions to handle and isolate cell nuclei and components thereof through use of lectin-coupled solid supports. The present disclosures further provide solid supports and kits and systems comprising the solid supports.

### CROSS-REFERENCES

This application claims the benefit of and priority to U.S. Provisional Patent Application No. 63/443,201, filed on Feb. 3, 2023, the entire contents of which are incorporated by reference herein.

### FIELD OF THE INVENTION

The present disclosure relates generally to methods and compositions for using lectins to isolate and manipulate cell nuclei.

### BACKGROUND OF THE INVENTION

Isolation of cell nuclei is useful in the fields of genetics, genomics, proteomics, and metabolomics and in a variety of biomedical applications. However, handling cell nuclei, including moving, immobilizing, isolating, enriching, or extracting, is difficult. Current methods to isolate or enrich cell nuclei are sub-optimal, often leading to contamination with other unwanted cell structures. As such, there is considerable room for improvement in methods that are effective and efficient for separation of cell nuclei from other cell structures.

### SUMMARY OF THE INVENTION

The present disclosure provides improved methods to handle and isolate cell nuclei and components thereof through targeting the sugar groups found on glycoproteins present in the nuclear membrane.

In one aspect, the present disclosure provides methods of associating cell nuclei to a solid support, comprising contacting nuclei in a sample with a solid support comprising one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc) present on glycoproteins in the nuclear membrane of the nuclei, thereby associating the nuclei to the solid support.

In some embodiments, the one or more lectin comprises Erythrina crista-galli lectin (ECL), Ricinus communis agglutinin (RCA), e.g., RCAI or RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin.

In some embodiments, the solid support is a bead.

In some embodiments, the solid support is a magnetic bead.

In some embodiments, the methods of associating cell nuclei to a solid support are used for isolating or extracting the nuclei from cells, cell lysate, and/or other non-nuclear cellular components.

In some embodiments, the nuclei are contacted with the solid support while in a sample comprising cells, cell lysate, and/or other non-nuclear cellular components.

In related embodiments, the solid support and associated nuclei are separated or removed from the sample, e.g., separated from cells, cell lysate, and/or other non-nuclear cellular components.

In related embodiments, the solid support and associated nuclei are separated or removed using a magnet.

In some embodiments, the methods of the present disclosure are used in transferring, moving, or washing cell nuclei.

In some embodiments, the methods further comprise separating or removing the solid support and associated nuclei from the sample, and optionally washing the nuclei.

In related embodiments, the methods further comprise separating or removing the solid support and associated nuclei using a magnet.

In some embodiments, the methods of the present disclosure are used for immobilizing the cell nuclei.

In some embodiments, the methods of the present disclosure are used for enriching the cell nuclei.

In some embodiments, the methods of the present disclosure are used for purifying the cell nuclei.

In certain embodiments, the methods do not comprise pelleting the nuclei.

In a related aspect, the present disclosure further provides methods of extracting nuclear proteins from cell nuclei, the method comprising:
(a) contacting nuclei with a first buffer to extract proteins from the nuclei;
(b) contacting the nuclei, e.g., after step (a), with a solid surface comprising one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc), thereby binding the nuclei to the solid support; and
(c) separating the buffer from the nuclei bound to the solid support, wherein the buffer comprises the nuclear proteins.

In some embodiments of the methods of extracting nuclear proteins from cell nuclei, the buffer is used to extract nucleoplasm proteins, chromatin-associated proteins, euchromatin-associated proteins, and/or heterochromatin-associated proteins.

In some embodiments of the methods of extracting nuclear proteins from cell nuclei, the buffer is an isotonic buffer used to extract nucleoplasm proteins.

In some embodiments of the methods of extracting nuclear proteins from cell nuclei, the buffer is a low salt buffer used to extract euchromatin-associated proteins.

In some embodiments of the methods of extracting nuclear proteins from cell nuclei, the buffer is a high salt buffer used to extract heterochromatin-associated proteins.

In some embodiments of the methods of extracting nuclear proteins from cell nuclei, the solid support comprises beads.

In some embodiments of the methods of extracting nuclear proteins from cell nuclei, the solid support comprises magnetic beads.

In some embodiments of the methods of extracting nuclear proteins from cell nuclei, the method comprises using a magnet to separate the solid support and associated nuclei from the buffer.

In some embodiments of the methods of extracting nuclear proteins from cell nuclei, the method comprises repeating steps (a)-(c) using a second buffer. In some embodiments, the first buffer is an isotonic buffer used to extract nucleoplasm proteins and the second buffer is a high salt buffer used to extract heterochromatin proteins.

In some related embodiments of the methods of extracting nuclear proteins from cell nuclei, the method comprises repeating steps (a)-(c) using a third buffer. In some embodiments, the first buffer is an isotonic buffer used to extract nucleoplasm proteins, the second buffer is a low salt buffer used to extract euchromatin proteins, and the third buffer is a high salt buffer used to extract heterochromatin proteins.

In related embodiments of the methods of extracting nuclear proteins from cell nuclei, the first buffer is an isotonic buffer used to extract nucleoplasm proteins, the second buffer is a low salt buffer used to extract euchromatin proteins, and the third buffer is a high salt buffer used to extract heterochromatin proteins.

In some embodiments of the methods of extracting nuclear proteins from cell nuclei, the method is a high-throughput method, wherein the nuclei are present in a first buffer within multiple wells of a sample plate, the solid support is a magnetic bead, and nuclei bound to the solid support are removed from the multiple wells using magnets.

In related embodiments of the high-throughput method, following removal from the multiple wells comprising the first buffer, the nuclei are placed into a second buffer within multiple wells of a sample plate, and the nuclei bound to the solid support are then removed from the multiple wells using magnets. In some embodiments, the first buffer is an isotonic buffer used to extract nucleoplasm proteins and the second buffer is a high salt buffer used to extract heterochromatin proteins.

In related embodiments of the high-throughput method, following removal from the multiple wells comprising the second buffer, the nuclei are placed into a third buffer within multiple wells of a sample plate, and the nuclei bound to the solid support are then removed from the multiple wells using magnets.

In related embodiments of the high-throughput method, the first buffer is an isotonic buffer used to extract nucleoplasm proteins, the second buffer is a low salt buffer used to extract euchromatin proteins, and the third buffer is a high salt buffer used to extract heterochromatin proteins. In other related embodiments of the high-throughput method, only one or only two buffers are used, which are each independently selected from an isotonic buffer used to extract nucleoplasm proteins, a low salt buffer used to extract euchromatin proteins, and a high salt buffer used to extract heterochromatin proteins. In particular embodiments, when any two or more buffers are used, they are used in the order listed.

In some embodiments of the methods of extracting nuclear proteins from cell nuclei, the method is a high-throughput method, wherein the nuclei are present in a first buffer within multiple wells of a sample plate, the solid support is a magnetic bead, and nuclei bound to the solid support are separated from the buffer using magnets and the buffer is transferred or removed.

In related embodiments of the high-throughput method, following removal of the first buffer from the multiple wells, a second buffer is added to the nuclei bound to the solid support within the multiple wells of a sample plate, and the nuclei bound to the solid support are separated from the buffer using magnets and the second buffer is transferred or removed. In some embodiments, the first buffer is an isotonic buffer used to extract nucleoplasm proteins and the second buffer is a high salt buffer used to extract heterochromatin proteins.

In related embodiments of the high-throughput method, following removal of the second buffer from the multiple wells, a third buffer is added to the nuclei bound to the solid support within the multiple wells of a sample plate, and the nuclei bound to the solid support are separated from the buffer using magnets and the third buffer is transferred or removed.

In related embodiments of the high-throughput method wherein the buffers are removed, the first buffer is an isotonic buffer to extract nucleoplasm proteins, the second buffer is a low salt buffer to extract euchromatin proteins, and the third buffer is a high salt buffer to extract heterochromatin proteins. In other related embodiments of the high-throughput method wherein the buffers are removed, only one or only two buffers are used, which are each independently selected from an isotonic buffer used to extract nucleoplasm proteins, a low salt buffer used to extract euchromatin proteins, and a high salt buffer used to extract heterochromatin proteins. In particular embodiments, when any two or more buffers are used, they are used in the order listed. In related embodiments of the high-throughput method wherein the buffer are removed, the first buffer is an isotonic buffer to extract nucleoplasm proteins, the second buffer is a high salt buffer to extract chromatin proteins.

The present disclosure further provides a solid support, wherein one or more lectin that binds to N-acetyllactosamine (LacNAc), galactose, N-acetylgalactosamine (GalNAc), and/or N-acetylglucosamine (GlcNAc) is coupled to a surface of the solid support.

In some embodiments of the solid support, the one or more lectin comprises Erythrina crista-galli lectin (ECL), Ricinus communis agglutinin (RCA), e.g., RCAI or RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin.

In some embodiments of the solid support, the one or more lectin comprises ECL.

In some embodiments of the solid support, the one or more lectin comprises RCA, optionally wherein the RCA is RCA120 or RCAI.

In some embodiments of the solid support, the solid support is a bead.

In some embodiments of the solid support, the solid support is a magnetic bead.

In some embodiments of the solid support, the one or more lectins are coupled to the surface of the solid support via a biotin-streptavidin conjugation.

The present disclosure further provides a kit, comprising:
(a) the solid support of the present disclosure; and
(b) one or more buffers suitable for extracting proteins from mammalian cell nuclei.

In some embodiments of the kit, the kit comprises one or more buffers suitable for lysing mammalian cells.

In some embodiments of the kit, the kit comprises one or more buffers selected from: an isotonic buffer, a low salt buffer, and a high salt buffer.

In a further aspect, the present disclosure further provides compositions of a system comprising:
(a) the solid support of the present disclosure, wherein the solid support is a magnetic bead;
(b) one or more buffers suitable for extracting proteins from mammalian cell nuclei; and
(c) one or more magnets.

In some embodiments of the system, the system comprises one or more buffers suitable for lysing cells.

In some embodiments of the system, the system comprises one or more buffers selected from: an isotonic buffer, a low salt buffer, and a high salt buffer.

In some embodiments of the system, the one or more magnets are present in a magnetic bead handling robot or liquid handling robot.

In some embodiments of the system, the system comprises a multi-well plate, wherein the one or more magnets are configured to fit into the wells of the multi-well plate or configured to sit below the multi-well plate to pull the magnetic beads to the bottom or side of the wells.

### BRIEF DESCRIPTION OF THE DRAWINGS

FIG. 1 provides glycomic mass spectrometry results in a volcano plot showing the enriched N-glycans in the cytosolic (containing the outer cell membrane) and LacNAc-containing glycans in the insoluble (containing the nuclear membrane) fractions of MM1.S cells that were subjected to chromatin extraction, respectively.

FIG. 2 provides a schematic of how lectin-conjugated magnetic beads can be used to extract proteins from cell nuclei. Nuclei are washed with buffers that contain increasing concentrations of NaCl, which separates the protein content into nucleoplasm, euchromatin (EUCH), and heterochromatin (HET) fractions.

FIGS. 3A-3D show representative images of nucleoplasm fractions taken using an EVOS FLoid imaging system. From left to right, the panels show images taken using blue light (BL), images taken using white light (brightfield; WL), and an overlaid image of the blue and white light images. FIG. 3A and FIG. 3B show images of the nucleoplasm fraction prepared using ConA-conjugated magnetic beads from two different samples. FIG. 3C shows images of the nucleoplasm fraction from a sample prepared using RCA120-conjugated magnetic beads. FIG. 3D shows images of the nucleoplasm fraction from a sample prepared using ECL-conjugated magnetic beads. FIG. 3E provides the results of protein quantification by 660 assay of the euchromatin fraction and heterochromatin fraction isolated from 4 million MMS. 1 cells using ConA, ECL, or RCA-conjugated magnetic beads. For each of the euchromatin fraction and heterochromatin fraction, the bars from left to right are results using magnetic beads conjugated to ConA, RCA, and ECL, respectively. Significant differences are indicated by “**.”

FIG. 4 shows the GO (gene ontology) enrichment analysis of proteins isolated using ConA, ECL, or RCA-conjugated magnetic beads, which demonstrates that RCA and ECL are superior for extracting nuclear proteins from cell nuclei. For each cell component, the bars from left to right are results using magnetic beads conjugated to ConA, RCA, and ECL, respectively.

FIG. 5 provides a schematic for high-throughput nuclear protein fractionation using a magnetic bead-handling robot with a magnetic head. The robot automates the use of lectin-conjugated magnetic beads to isolate separate nuclear fractions such as nucleoplasm, euchromatin, and heterochromatin fractions.

FIGS. 6A-6C show the peptides (FIG. 6A) and proteins (FIG. 6B) that were detected by LC-MS from three biological replicates of chromatin extracted from THP-1 nuclei using ECL-conjugated magnetic beads. FIG. 6C shows the coefficient of variation (CV) of protein abundances across the three biological replicates in this experiment.

FIGS. 7A-7F show protein abundances of bromodomain-containing protein 4 (BRD4) (FIG. 7A), bromodomain-containing protein 3 (BRD3) (FIG. 7B), bromodomain-containing protein 2 (BRD2) (FIG. 7C), menin (MEN1) (FIG. 7D), histone H4 (H4) (FIG. 7E), and beta-actin (ACTB) (FIG. 7F) in three drug treatment conditions from three replicates of LC-MS experiments analyzing protein fractions that were extracted from THP-1 cell nuclei by automation with the KingFisher™ robot using ECL-conjugated magnetic beads.

### DETAILED DESCRIPTION OF THE INVENTION

In certain embodiments, the present disclosure provides methods of associating cell nuclei to a solid support and uses thereof to isolate or extract cell nuclei, nuclear subfractions, cell lysate and/or other non-nuclear cellular components. In some embodiments, the methods disclosed herein are used to move, transfer, or wash cell nuclei. Aspects of the methods disclosed herein are based on the identification of lectins that specifically or preferentially bind to sugar groups present on glycans or glycoproteins present in or on the nuclear membrane, and the development of methods of coupling these lectins to a solid support, thus providing a means of selectively binding nuclei and nuclear membrane to the solid support. In some embodiments, one or more lectin is coupled to the solid support. In some embodiments, the one or more lectin binds to one or more glycans or glycoproteins found on the nuclear membrane. In some embodiments, the one or more lectin binds to one or more glycans enriched on the nuclear membrane.

### Lectins and Glycans

Lectins are a class of proteins that bind to certain sugar groups, or glycans. Concanavalin A (ConA), a lectin that binds selectively to an N-glycan called mannose, has been shown through staining to bind to the nuclear membrane (see Monneron et. al., Extensive Binding of Concanavalin A to the Nuclear Membrane, FEBS Lett. 1974, 42 (2), 209-213). However, the evidence of N-glycans actually occurring on the surface of the nuclear membrane is limited (see Stanley et. al., N-Glycans, Cold Spring Harbor Laboratory Press, 2022).

The present disclosure provides data showing that mannoses are enriched on the outer cell membrane and not the nuclear membrane. The nuclear membrane is instead enriched with glycoproteins containing glycans such as N-acetyllactosamine (LacNAc), galactose, N-acetylgalactosamine (GalNAc), and N-acetylglucosamine (GlcNAc). Erythrina crista-galli lectin (ECL) and Ricinus communis agglutinin (RCA), e.g., RCA120, are lectins that bind to galactose, N-acetylgalactosamine (GalNAc), and N-acetyllactosamine (LacNAc). ECL and RCA, e.g., RA120, are superior in specificity and binding capability to ConA in binding, isolating, or enriching cell nuclei, thereby reducing loss of cell nuclei during sample processing and increasing purity of isolated cell nuclei or fractions or components thereof when used in the methods described herein. Additionally, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and Wisteria floribunda lectin also have superior binding specificity for N-acetyllactosamine (LacNAc), galactose, N-acetylgalactosamine (GalNAc), and/or N-acetylglucosamine (GlcNAc) and can also be used in the methods described herein.

Lectins, such as ECL and RCA, that bind to glycans enriched on the nuclear membrane may be coupled to a solid support to facilitate handling, isolating, and/or extracting cell nuclei and subfractions thereof. In some embodiments, the one or more lectin coupled to the solid support comprises Erythrina crista-galli lectin (ECL), Ricinus communis agglutinin (RCA), e.g., RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectins comprise both Erythrina crista-galli lectin and Ricinus communis agglutinin. In some embodiments, the lectin is Erythrina crista-galli lectin. In some embodiments, the lectin is Ricinus communis agglutinin. In certain embodiments, the Ricinus communis agglutinin is RCA120. In some embodiments, the one or more glycan is enriched on the nuclear membrane. In some embodiments, the one or more glycan comprises one or more of N-acetyllactosamine (LacNAc), galactose, N-acetylgalactosamine (GalNAc), and/or N-acetylglucosamine (GlcNAc). In some embodiments, the one or more glycans is a combination of any of the glycans selected from a list consisting of N-acetyllactosamine (LacNAc), galactose, N-acetylgalactosamine (GalNAc), and/or N-acetylglucosamine (GlcNAc).

### Solid Support

As described herein, a “solid support” comprises any flexible or rigid substrate or material. In certain embodiments, the solid support comprises any flexible or rigid substrate or material onto which one or more binding agent may be applied. In some embodiments, the solid support is or comprises a planar surface. In some embodiments, the solid support has or comprises a tubular surface. In some embodiments, the solid support has or comprises a circular or spherical surface. In some embodiments, the solid support is a bead. In some embodiments, the solid support is magnetic. In some embodiments, the solid support is a magnetic bead. In some embodiments, the solid support is biocompatible, wherein it is not toxic to living cells or tissues.

In some embodiments, the surface (or a portion thereof) or substrate of the solid support to which the one or more lectin is bound comprises a material or reagent different from that of the rest of the solid support.

Lectins may be directly or indirectly bound to the surface of the solid support. In some embodiments, the solid support comprises a functionalized surface for coupling to proteins. In some embodiments, the solid support has a functionalized surface for coupling to lectins. In some embodiments, the solid support has a surface comprising streptavidin (streptavidin-coated). In some embodiments, one or more lectin that binds to LacNAc, galactose, GalNAc, and/or GlcNAc is coupled to a surface of the solid support. In some embodiments, the one or more lectins are coupled to the surface of the solid support via a biotin-streptavidin conjugation. In some embodiments, the one or more lectin is biotinylated and binds to the surface of a solid support that is streptavidin-coated via biotin-streptavidin conjugation. In some embodiments, the one or more lectin comprises Erythrina crista-galli lectin (ECL), Ricinus communis agglutinin (RCA), e.g., RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectin is Erythrina crista-galli lectin (ECL). In some embodiments, the lectin is Ricinus communis agglutinin (RCA).

### Methods

The methods disclosed herein are used to handle cell nuclei, including moving, immobilizing, isolating, enriching, or extracting, cell nuclei. In certain aspects, the methods are used to isolate nuclear proteins. Generally, the methods include contacting cells or components or subfractions thereof (referred to as samples or cell samples) with lectins coupled to a solid support. In some embodiments, the solid support is magnetic, and the components of the sample bound to the solid support, e.g., nuclei, may be moved or transferred by a magnet. In some embodiments, the sample comprises live cells. In some embodiments, the sample comprises cell nuclei.

### Cells

The cells may be from a variety of sources. For example, the cells may be cell lines, including primary cell lines, or they may be obtained from a tissue, organ, or organism, e.g., mammalian cells. Cells may also be present in cultured organoids, e.g., in vitro organoids produced from cells obtained from a cell, tissue, organ, or organism, e.g., a mammal.

In some embodiments, the cells are mammalian cells or are obtained from a mammal. The mammalian cells may be any cell type. In some embodiments, the mammalian cell is an epithelial cell, a connective tissue cell, a hormone secreting cell, a nerve cell, a skeletal muscle cell, a blood cell, an immune system cell, or a stem cell.

In certain embodiments, the cells are obtained from blood, serum, urine, stool, saliva, lymph fluid, cerebrospinal fluid, synovial fluid, cystic fluid, ascites, pleural effusion, amniotic fluid, chorionic villus sample, vaginal fluid, interstitial fluid, nasal swab sample, buccal swab sample, sputum, bronchial lavage, Pap smear sample, or ocular fluid. The cell sample may comprise cells obtained from a blood sample, an aspirate sample, or a smear sample. Cells may be obtained from a biopsy sample.

In certain embodiments, the cells are derived from a cell line. Illustrative cell lines include, but are not limited to, 293A cell line, 293 FT cell line, 293F cell line, 293 H cell line, HEK 293 cell line, CHO DG44 cell line, CHO-S cell line, CHO-K1 cell line, Expi293F™ cell line, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cell line, FreeStyle™ CHO-S cell line, GripTite™ 293 MSR cell line, GS-CHO cell line, HepaRG™ cell line, T-REX™ Jurkat cell line, Per.C6 cell line, T-REx™-293 cell line, T-REX™-CHO cell line, T-REX™-HeLa cell line, NC-HIMT cell line, and PC12 cell line.

The cells may comprise healthy and/or diseased or damaged cells. For example, in certain embodiments, the cells are obtained from a healthy mammal or from a mammal diagnosed with a disease or disorder, such as, e.g., a cancer or tumor, an inflammatory disease or disorder, an immune disease or disorder, a genetic disease or disorder, a metabolic disease or disorder, a cardiac disease or disorder, ischemia or reperfusion injury, or an infection, e.g., infection by bacteria, virus, fungi, etc. Cells may be obtained from a healthy or a diseased tissue or organ.

A cell sample may comprise cancerous cells. The cancerous cells may be derived from a cancer, e.g., a solid tumor or a hematologic malignancy.

In some embodiments, the cancerous cell sample may comprise cells obtained from a solid tumor. In some embodiments, the solid tumor may include a sarcoma or a carcinoma.

Illustrative sarcoma cell samples may include, but are not limited to, cell samples obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (WE), malignant fibrous histiocytoma (WE) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epithelioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Illustrative carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some embodiments, the cancerous cell sample may comprise cells obtained from a hematologic malignancy. In some embodiments, the hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Illustrative B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Illustrative T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some embodiments, the cell sample may comprise circulating tumor cells. In some embodiments, a circulating tumor cell sample may comprise lymphoma cells, fetal cells, apoptotic cells, epithelial cells, endothelial cells, stem cells, progenitor cells, mesenchymal cells, osteoblast cells, osteocytes, hematopoietic stem cells, foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a lung, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

In some embodiments, a cell sample may be a peripheral blood mononuclear cell sample or comprise peripheral blood mononuclear cells.

In some embodiments, a cell sample may comprise cells of a tumor cell line. Illustrative tumor cell lines include, but are not limited to, cell samples from tumor cell lines such as MM1.S, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

Cell samples (such as biopsy samples) may be obtained from a mammal by any suitable means of obtaining the sample using well-known and routine clinical methods. For example, surgical procedures, and procedures for drawing and processing tissue samples such as from a needle aspiration biopsy are well-known and may be employed to obtain a sample for use in the methods provided. Typically, for collection of such a tissue sample, a thin hollow needle is inserted into a mass such as a tumor mass for sampling of cells that, after being stained, will be examined under a microscope.

### Cell Processing and Isolation of Nuclei

Cells may be harvested, washed, and/or lysed, e.g., to allow separation of nuclei from other cellular material. Methods of harvesting, washing, and lysing cells, e.g., mammalian cells, are well known and available in the art, and any method may be used. In particular embodiments, the method lyses the cell membrane but leaves the nuclei intact.

In preparation for cell nuclei isolation, cells may be harvested by centrifugation, and washed, e.g., with PBS, and then re-centrifuged to generate a cell pellet. In some embodiments, cells are centrifuged at about 400 RCF for about 5 minutes at about 4° C. In some embodiments, the cells are washed with ice cold 1× PBS. In some embodiments, the cells are washed more than one time with PBS.

Harvested cells may be lysed, e.g., to release cellular components such as nuclei. In certain embodiments, cells, e.g., washed and pelleted cells, are resuspended in a first suspension buffer, e.g., a cell lysis buffer. The resuspended cells may be lysed by this first suspension buffer through means known in the art. In certain embodiments, this first suspension buffer comprises a detergent that lyses the cells. The detergent may be ionic or non-ionic. In certain embodiments, the detergent comprises NP40. In certain embodiments, the detergent comprises SDS, TritonX100, and/or Tween20. The detergent may be a mass-spectrometry acid-labile detergent. In some embodiments, the concentration of the detergent in the first suspension buffer is up to 4%. In some embodiments, the concentration of the detergent ranges from about 0.01% to about 10%, from about 0.01% to about 4% or from about 0.1% to about 4%, or the concentration of the detergent may be any integer falling within any of these ranges. For example, the detergent may be at a concentration of 0.01% to 0.1%. In some embodiments, the first suspension buffer is NEB. In some embodiment the first suspension buffer is a Nuclear Extraction Buffer (NEB) comprising about 0.1% Triton X-100. In certain embodiments, the NEB comprises about 20 mM HEPES, about 10 mM KCl, about 20% Glycerol, about 1 mM MnCl₂, and about 0.1% Triton X-100. The cells are contacted with the first suspension buffer for a time sufficient to lyse the cells. In some embodiments, the period of contact is at least 10 minutes. In certain embodiments, other methods may be used to lyse the cells, such as, e.g., sonication or physical cutting or mincing. In some embodiments, cell lysis is performed on a cold block, on wet ice, or at 4° C.

In some embodiments, after cell lysis, the lysed cell sample, e.g., which contains nuclei, is contacted with lectins bound to a solid support. In some embodiments, the lectins include one or more lectin that binds to LacNAc, galactose, GalNAc, and/or GlcNAc. In some embodiments, the one or more lectins are coupled to the surface of the solid support via a biotin-streptavidin conjugation. In some embodiments, the one or more lectin is biotinylated and binds to the surface of a solid support that is streptavidin-coated via biotin-streptavidin conjugation. In some embodiments, the one or more lectin comprises Erythrina crista-galli lectin (ECL), Ricinus communis agglutinin (RCA), e.g., RCA!20, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectin is Erythrina crista-galli lectin (ECL). In some embodiments, the lectin is Ricinus communis agglutinin (RCA). In some embodiments, the solid support is a magnetic bead. The sample and lectins bound to the solid support may be incubated for a time and under conditions sufficient to allow binding of the lectins to the nuclei present in the lysed cell sample. The unbound components of the lysed cell sample (supernatant) may then be separated from the solid support-bound nuclei. In some embodiments, this supernatant can be retained as the Cytosolic Fraction. In certain embodiments, the solid support, e.g., a bead, may be pelleted, and the supernatant removed. In certain embodiments, the solid support, e.g., a magnetic bead, may be subjected to a magnet, in order to hold the solid support in place, while the supernatant is removed, e.g., through the side of a tube holding the lysed cell sample and solid support. In certain embodiments, a magnet may be introduced into the lysed cell sample containing the solid support, and the solid support with bound nucleic is then removed from the remainder of the sample (supernatant).

In some embodiments, after cell lysis, the lysed cell sample, e.g., which contains nuclei, is centrifuged. In some embodiments, the samples are centrifuged at about 400 RCF for about 5 minutes at about 4° C. The supernatant is then removed. In some embodiments, this supernatant is discarded. In some embodiments, this supernatant can be retained as the Cytosolic Fraction. The remaining pellet is enriched for cell nuclei. The remaining pellet may be resuspended in a buffer or suspension fluid and nuclei may then be bound to a solid support by adding the solid support to the suspension of nuclei. The nuclei may then be further isolated from other cellular components by contacting the suspension with lectins bound to a solid support, in a manner similar to described above or herein.

Isolated nuclei may be washed and further manipulated, processed, and/or analyzed.

### Isolation of Nuclear Proteins

In one application, nuclear proteins may be isolated from nuclei prepared using lectins bound to a solid support as described herein. In addition, or alternatively, nuclear proteins may be isolated from nuclei using lectins bound to a solid support as described herein. In some embodiments, the lectins include one or more lectin that binds to LacNAc, galactose, GalNAc, and/or GlcNAc. In some embodiments, the one or more lectins are coupled to the surface of the solid support via a biotin-streptavidin conjugation. In some embodiments, the one or more lectin is biotinylated and binds to the surface of a solid support that is streptavidin-coated via biotin-streptavidin conjugation. In some embodiments, the one or more lectin comprises Erythrina crista-galli lectin (ECL), Ricinus communis agglutinin (RCA), e.g., RCA120, Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin. In some embodiments, the lectin is Erythrina crista-galli lectin (ECL). In some embodiments, the lectin is Ricinus communis agglutinin (RCA). In some embodiments, the solid support is a magnetic bead.

In certain embodiments, the cell nuclei are suspended in or contacted with a suspension buffer comprising a salt concentration suitable for extraction of nuclear proteins from the cell nuclei. For example, the nuclear proteins may include, e.g., nucleoplasm-enriched proteins, euchromatin-enriched proteins, and/or heterochromatin-enriched proteins. The cell nuclei may be bound to lectins on a solid support when they are suspended in a suspension buffer.

In certain embodiments, after removal of the supernatant containing cytosolic proteins, the pellets are re-suspended in a suspension buffer, wherein the concentration of the salt is used to selectively extract one or more subset of nuclear proteins, e.g., one or more of: nucleoplasm-enriched proteins, euchromatin-enriched proteins, and/or heterochromatin-enriched proteins. In one embodiment, the nuclei may be re-suspended and incubated in an isotonic buffer to extract nucleoplasm-enriched proteins, which may include centrifugation to collect supernatant as a Nucleoplasm Fraction and/or insoluble chromatin. In one embodiment, the nuclei may be re-suspended and incubated in a low salt buffer to extract nucleoplasm-enriched proteins and/or euchromatin-associated proteins, which may include centrifugation to collect supernatant as a Nucleoplasm and Euchromatin Fraction and insoluble chromatin. In one embodiment, the nuclei may be re-suspended and incubated in a high salt buffer to extract nucleoplasm-enriched proteins, euchromatin-associated proteins and/or heterochromatin-associated proteins, which may include centrifugation to collect supernatant as a Heterochromatin Fraction.

In some embodiments, the suspension buffer is an isotonic buffer, a low salt buffer, or a high salt buffer.

An isotonic buffer comprises low salt concentration (e.g., 5-20 mM Na⁺). In some embodiments, an isotonic buffer comprises a NaCl concentration of about 15 mM. In some embodiments, an isotonic buffer has approximately physiological salt concentrations, and in certain embodiments, an isotonic buffer comprises a salt concentration of about 10 mM to about 20 mM. In some embodiments, an isotonic buffer comprises a NaCl concentration of about 10 mM to about 20 mM. In some embodiments, an isotonic buffer comprises a NaCl concentration of about 15 mM. In some embodiments, the second suspension buffer is EDTA-free Isotonic Buffer. In some embodiment, the EDTA-free Isotonic buffer comprises or consists of about 10 mM Tris pH 8.0, about 15 mM NaCl, and about 60 mM KCl. In some embodiments, the isotonic buffer is EDTA-free. In some embodiments, the EDTA-free Isotonic buffer comprises or consists of about 10 mM Tris pH 8.0, about 15 mM NaCl, and about 60 mM KCl.

A low salt buffer comprises a medium salt concentration (e.g., 20-400 mM or 25-400 mM Na⁺). In some embodiments, a low salt buffer has a salt, e.g., NaCl, concentration of about 100 mM to about 300 mM. In some embodiments, a low salt buffer has a salt, e.g., NaCl, concentration of about 100 mM to about 200 mM. In some embodiments, the low salt buffer has a NaCl concentration of about 150 mM or about 160 mM. In some embodiments, the low salt buffer is a Euchromatin Buffer. In some embodiments, the Euchromatin Buffer comprises about 10 mM Tris pH 8.0, about 250 mM NaCl, and about 1 mM EDTA pH 8.0.

A high salt buffer comprises a high salt concentration (e.g., >400 mM Na⁺, about 400 to 1000 mM Na⁺, about 401 to 1000 mM Na⁺, about 450 to 1000 mM Na⁺, about 450 to 8000 mM Na⁺, about 400-800 mM Na⁺, or about 401-800 mM Na⁺). In some embodiments, a high salt buffer has a salt, e.g., NaCl, concentration of about 400 mM to about 800 mM. In some embodiments, the high salt buffer has a NaCl concentration of about 450 mM, about 500 mM, about 550 mM, about 600 mM, about 650 mM, or about 700 mM. In some embodiments, the high salt buffer has a NaCl concentration of about 600 mM. In some embodiment, the high salt buffer is a Heterochromatin Buffer. In some embodiment, the Heterochromatin Buffer comprises about 10 mM Tris pH 8.0, about 600 mM NaCl, and about 1 mM EDTA PH 8.0.

In certain embodiments, a high salt buffer further comprises one or more detergent. In particular embodiments, the one or more detergent comprises Triton X100, NP-40, or sodium dodecyl sulfate (SDS). The detergent may be a mass-spectrometry acid-labile detergent. In some embodiments, the concentration of the detergent in the first suspension buffer is up to 4%. In some embodiments, the concentration of the detergent ranges from 0.01% to 4% or from 0.1% to 4%. For example, the detergent may be at a concentration of 0.01% to 0.1%. In particular embodiments, the one or more detergent is present in the high salt buffer at a concentration of 0.1% to 1.0%. In one embodiment, a high salt buffer comprises about 750 mM salt, e.g., NaCl, and about 0.1% to 1.0% of Triton X100, NP-40, or sodium dodecyl sulfate (SDS).

In certain embodiments, following suspension in or contacting with a suspension buffer for a time sufficient to extract nuclear proteins from the nuclei, the remaining nuclei are separated from the supernatant, which now contains the nuclear proteins, e.g., through the use of the solid support. The solid support may be removed from the supernatant, or the supernatant may be removed from the solid support, e.g., through the use of a magnet to hold or move a magnetic bead solid support to which the nuclei and/or nuclear membranes are bound.

In certain embodiments, any or all of the buffers and solutions used to lyse cells and/or isolate nuclear proteins do not comprise polycations.

In certain embodiments, the cell nuclei are re-suspended in or contacted with a sequential series of suspension solutions comprising increasing salt concentrations, with each suspension solution used for selective extraction of various subsets of nuclear proteins, including, e.g., nucleoplasm-enriched proteins, euchromatin-enriched proteins, and/or heterochromatin-enriched proteins. For example, (1) the nuclei may be re-suspended in, contacted with, and/or incubated in an isotonic buffer to extract nucleoplasm-associated proteins, followed by separation of the nuclei from the supernatant referred to as a Nucleoplasm Fraction and which contains nucleoplasm-associated proteins; (2) the remaining nuclei and/or insoluble chromatin resulting from (1) may be re-suspended in, contacted with, and/or incubated in a low salt buffer to extract euchromatin-associated proteins, followed by separation of the nuclei and/or insoluble chromatin from the supernatant referred to as a Euchromatin Fraction and which contains euchromatin-associated proteins; and (3) the nuclei and/or insoluble chromatin resulting from (2) may be re-suspended in, contacted with, and/or incubated in a high salt buffer to extract heterochromatin-associated proteins, following by separation of the nuclei and/or insoluble chromatin from the supernatant referred to as a Heterochromatin Fraction, which contains heterochromatin-associated proteins. It is further understood that the samples do not necessarily have to be resuspended in each of the isotonic, low salt, and high salt buffers; instead; the method may be performed using one or any two of these buffers, e.g., to selectively enrich for nuclear proteins associated with desired nuclear fraction(s). For example, in certain embodiments, (1) the nuclei may be re-suspended in, contacted with, and/or incubated in an isotonic buffer to extract nucleoplasm-associated proteins, followed by separation of the nuclei from the supernatant referred to as a Nucleoplasm Fraction and which contains nucleoplasm-associated proteins; (2) the remaining nuclei and/or insoluble chromatin resulting from (1) may be re-suspended in, contacted with, and/or incubated in a high salt buffer to extract heterochromatin-associated proteins, following by separation of the nuclei and/or insoluble chromatin from the supernatant referred to as a Heterochromatin Fraction, which contains heterochromatin-associated proteins. The nuclei and insoluble chromatin may be incubated in the various buffers for times and under conditions known in the art or described herein.

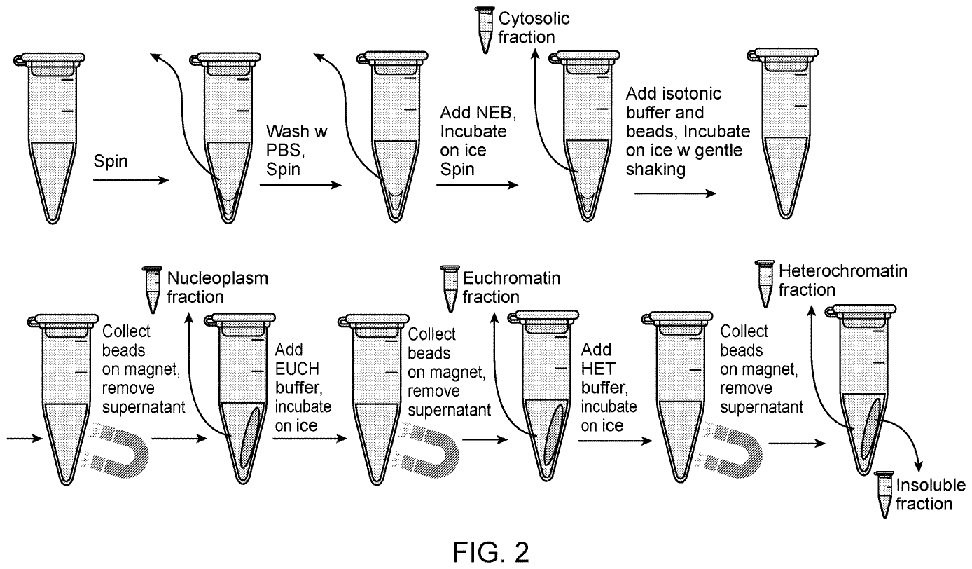

According to any of the methods, the nuclei may be separated from the supernatant via the solid support, e.g., as shown in FIG. 2.

The suspension solutions and buffers may comprise a salt. The salt may comprise an alkali or alkaline earth salt, such as a lithium salt, a sodium salt, a potassium salt, a rubidium salt, a magnesium salt, a calcium salt, or a strontium salt. The salt may comprise a halide salt, such as a fluoride salt, a chloride salt, a bromide salt, or an iodide salt. A salt may comprise an alkali halide salt, such as lithium fluoride, lithium chloride, lithium bromide, lithium iodide, sodium fluoride, sodium chloride, sodium bromide, sodium iodide, potassium fluoride, potassium chloride, potassium bromide, or potassium iodide. The salt may comprise an alkaline earth halide salt, such as magnesium fluoride, magnesium chloride, magnesium bromide, magnesium iodide, calcium fluoride, calcium chloride, calcium bromide, calcium iodide, strontium fluoride, strontium chloride, strontium bromide, or strontium iodide. The salt may comprise an ammonium salt. The salt may comprise a transition metal salt. The salt may comprise an acetate, benzoate, carbonate, chromate, citrate, cyanide, hypochlorite, chlorite, chlorate, perchlorate, dichromate, dihydrogen phosphate, bicarbonate, bisulfate, hydrogen phosphate, hydroxide, nitrite, nitrate, peroxide, permanganate, phosphate, sulfite, or sulfate salt. In some embodiments, the salt is sodium chloride.

In some embodiments, the resuspended cell nuclei may be incubated in the suspension solution or buffer. In certain embodiments, the nuclei may be incubated at a temperature of about 0° C., about 4° C., about 10° C., about 20° C., about 40° C., or about 100° C. The nuclei may be incubated at a temperature that is within a range defined by any two of the preceding values. In certain embodiments, the nuclei may be incubated for a period of about 1 minute, about 3 minutes, about 5 minutes, about 10 minutes, about 20 minutes, about 30 minutes, about 50 minutes, or about 100 minutes. The cell nuclei may be incubated for a period that is within a range defined by any two of the preceding values.

In some embodiments, after removal of the cytosolic fraction, the pellet containing the nuclei is then resuspended in a second suspension buffer. In some embodiments, the second suspension buffer is an isotonic buffer. In some embodiments, an isotonic buffer has approximately physiological salt concentrations, and in certain embodiments, an isotonic buffer comprises a salt concentration of about 10 mM to about 20 mM. In some embodiments, an isotonic buffer comprises a NaCl concentration of about 10 mM to about 20 mM. In some embodiments, an isotonic buffer comprises a NaCl concentration of about 15 mM. In some embodiments, the second suspension buffer is EDTA-free Isotonic Buffer. In some embodiment, the EDTA-free Isotonic buffer comprises or consists of about 10 mM Tris pH 8.0, about 15 mM NaCl, and about 60 mM KCl.

The sample resuspended in the second suspension buffer is then added to and/or thoroughly mixed with the lectin-coupled solid support. For example, activated lectin-conjugated magnetic beads are added to the sample with the pellet and the EDTA-free Isotonic Buffer and mixed by pipetting up and down. In some embodiments, activated lectin-conjugated magnetic beads are added at a volume of 20 μl per million cells.

The samples are then contacted with the lectin-coupled solid support for a time sufficient for the cell nuclei to bind to the lectin-coupled solid support. In some embodiments, the contact period is at least 5, 10, or 20 minutes. In some embodiments, the sample is subjected to gentle shaking during the contact period. In some embodiments, the sample is placed on wet ice or kept at 4° C. during the contact period.

After the cell nuclei are bound to the solid support, the supernatant is separated from the cell nuclei bound to the solid support through centrifugation or other means known in the arts. For example, cell nuclei bound to beads can be centrifuged. In some embodiments, cell nuclei bound to lectin-conjugated magnetic beads are placed onto a magnetic rack and allowed to sit for a sufficient amount of time for the magnetic beads to pellet. The supernatant is then removed carefully and retained as the Nucleoplasm Fraction. The nucleoplasm fraction is enriched for nuclear proteins.

In some embodiments, the sample containing the pellet is then resuspended in a third suspension buffer. In some embodiments, the third suspension buffer is a low salt buffer. In some embodiments, a low salt buffer has a salt, e.g., NaCl, concentration of about 100 mM to about 200 mM. In some embodiments, the low salt buffer has a NaCl concentration of about 150 mM or about 160 mM. In some embodiments, the third suspension buffer is Euchromatin Buffer. In some embodiments, the Euchromatin Buffer comprises 10 mM Tris pH 8.0, 250 mM NaCl, and 1 mM EDTA pH 8.0. In some embodiments, the resuspended pellet is incubated in the third suspension buffer 4° C. for 15 minutes.

The supernatant is then separated from the pellet containing cell nuclei, cell nuclei debris, insoluble chromatin, and/or insoluble protein bound to the solid support through centrifugation or other means known in the arts as described above. For example, cell nuclei and/or insoluble chromatin bound to lectin-conjugated magnetic beads are placed onto a magnetic rack and allowed to sit for a sufficient amount of time for the magnetic beads to pellet. The supernatant is then removed carefully and retained as the Euchromatin Fraction. The euchromatin fraction is enriched for euchromatin-associated proteins.

In some embodiments, the sample containing the pellet is then resuspended in a fourth suspension buffer. In some embodiments, the fourth suspension buffer is a high salt buffer. In some embodiments, a high salt buffer has a salt, e.g., NaCl, concentration of about 400 mM to about 800 mM. In some embodiments, the high salt buffer has a NaCl concentration of about 600 mM. In some embodiments, the fourth suspension buffer is Heterochromatin Buffer. In some embodiments, the Heterochromatin Buffer comprises 10 mM Tris pH 8.0, 600 mM NaCl, and 1 mM EDTA pH 8.0. In some embodiments, the resuspended pellet is incubated in the fourth suspension buffer 4° C. for 15 minutes. The supernatant is then separated from the pellet containing cell nuclei, cell nuclei debris, insoluble chromatin, and/or insoluble proteins bound to the solid support through centrifugation or other means known in the arts as described above. For example, cell nuclei and/or insoluble chromatin bound to lectin-conjugated magnetic beads are placed onto a magnetic rack and allowed to sit for a sufficient amount of time for the magnetic beads to pellet. The supernatant is then removed carefully and retained as the Heterochromatin Fraction. The heterochromatin fraction is enriched for heterochromatin-associated proteins. The remaining pellet contains the insoluble fraction and may be stored for future use. The insoluble fraction contains cell debris, chromatin, and can contain insoluble chromatin-associated proteins and may be extracted and analyzed as described in the methods herein. The insoluble fraction may also be useful for analyzing histone post-translational modifications, or potentially isolating RNA or DNA for further analysis of the sample.

In other related embodiments, the sample containing the pellet is suspended in ony one or any two of the second, third, and fourth suspension buffer.

In some embodiments, after cell lysis, the samples containing the isolated nuclei are then added to and/or thoroughly mixed with the lectin-coupled solid support. For example, activated lectin-conjugated magnetic beads are added to the samples and mixed by pipetting up and down. In some embodiments, activated lectin-conjugated magnetic beads are added at a volume of 20 μl per million cells. The samples are then contacted with the lectin-coupled solid support for a time sufficient for the cell nuclei to bind to the lectin-coupled solid support. In some embodiments, the contact period is at least 5, 10, or 20 minutes. In some embodiments, the sample is subjected to gentle shaking during the contact period. In some embodiments, the sample is placed on wet ice or kept at 4° C. during the contact period. After the cell nuclei are bound to the solid support, the supernatant is separated from the cell nuclei bound to the solid support through centrifugation or other means known in the arts. For example, cell nuclei bound to beads can be centrifuged. In some embodiments, cell nuclei bound to lectin-conjugated magnetic beads are placed onto a magnetic rack and allowed to sit for a sufficient amount of time for the magnetic beads to pellet. The supernatant is then removed carefully, and the cell pellet is resuspended in a suitable buffer, such as an isotonic buffer. The supernatant is then separated and discarded, and the enriched cell nuclei may be retained as the Nuclear Fraction and used for downstream applications as described herein.

In particular embodiments, any of the methods disclosed herein comprises one or more of: (1) using detergents (NP-40) for nuclear isolation to ensure that the proteins are not denatured during the process; (2) use of EDTA and/or protease inhibitors to prevent protein degradation during the process; (3) not using buffers with polycations, which interfere with protein analysis; and (4) sonication or removal of DNA/RNA by enzymatic processes.

### High-Throughput Processing

High-throughput methods are often more cost-effective and increase efficiency when a large number of samples are analyzed. The use of lectin-coupled solid supports for handling cell nuclei and the related methods described herein can be carried out in any compatible high-throughput format, such as with the use of multi-well plates. For example, multiple samples coupled to lectin-conjugated beads may be placed within multiple wells of a 96-well plate to be centrifuged and washed. In some embodiments, the lectin-conjugated beads are magnetic and a magnet compatible with the multi-well plate is used to collect or bind the magnetic beads in order to pellet, move, or transfer the sample. In some embodiments, the magnet immobilizes the nuclei or nuclear membrane bound to the magnetic beads, while the supernatant is removed or while suspension buffer is added. In some embodiments, the magnet binds to the nuclei and/or nuclear membrane bound to the magnetic beads and used to move or transfer the samples into a vessel, such as a separate multi-well plate. In some embodiments, an automation instrument may be used to process samples as described herein. In some embodiments, the instrument can be an automated liquid handler. In some embodiment, the instrument can be a magnetic bead handling robot. In some embodiments, the instrument can be an automated sample purification or extraction system, such as the Thermo Scientific KingFisher™ Purification System. In some embodiments, one or more of the steps to isolate or extract the cell nuclei and/or fractions thereof can be performed using the automated system. In some embodiments, all the isolation and extraction steps are performed using the automated system.

In some embodiments, the high throughput method comprises multiple samples of cell nuclei that are present in a buffer within multiple wells of a sample plate and a solid support of magnetic beads, wherein the cell nuclei bound to the solid support are removed from the multiple wells using magnets. In some embodiments, the cell nuclei bound to the solid support are then placed into a second buffer within multiple wells of a sample plate, wherein the second buffer may be the same or a different buffer from the preceding buffer. In some embodiments, the steps of removing the cell nuclei bound to the solid support from the multiple wells using magnets and placing them into a second buffer may be repeated one or more times. In some embodiments, the cell nuclei bound to the solid support, wherein the solid support is lectin-conjugated magnetic beads, are removed from the multiple wells using magnets and sequentially placed into and removed from a series of buffers, wherein the buffers comprise an isotonic buffer to extract nucleoplasm proteins, a low salt buffer to extract euchromatic proteins, and a high salt buffer to extract heterochromatin proteins.

### Kits

The lectin-coupled solid support described herein may be used as a component of a kit for isolation of cell nuclei. In some embodiments, the kit comprises one or more of the solid supports as described in the present disclosure. In some embodiments, the kit comprises lectin-conjugated beads. In some embodiments, the kit comprises lectin-conjugated magnetic beads. In some embodiments, the kit comprises one or more buffers suitable for lysing mammalian cells. In some embodiments, the kit comprises Nuclear Extraction Buffer. In some embodiments, the kit comprises one or more buffers selected from: an isotonic buffer, a low salt buffer, and a high salt buffer. In some embodiments, the kit comprises one or more buffers selected from: EDTA-free Isotonic Buffer, Euchromatin Buffer, and Heterochromatin Buffer. In some embodiments, the kit comprises a magnet.

### Systems

The lectin-coupled, magnetic solid support, the one or more buffers as described herein, and/or one or more magnets can form the components of a system used to automatically isolate cell nuclei. For example, a robot that is capable of handling magnetic beads can be programmed to perform the steps of binding, moving, and/or transferring the samples attached to the magnetic beads from one vessel to another. In some embodiments, the system comprises one or more multi-well plate and the one or more magnets are configured to fit into the wells of the multi-well plate. In some embodiments, the one or more magnets are configured to fit around the outside of the wells of the multi-well plate to pellet samples attached to the magnetic beads. The supernatant is then removed and retained (transferred to a separate multi-well plate) or discarded. Optionally, the multi-well plate is then removed from the magnet, a buffer is dispensed into the wells and mixed with the samples and the plate placed on the magnet. The supernatant can then be removed as described and the steps repeated with one or more buffers. In some embodiments, one or more buffers is selected from; and isotonic buffer, a low salt buffer, and a high salt buffer. In some embodiments, the one or more buffers is selected from: Nuclear Extraction Buffer, EDTA-free Isotonic Buffer, Euchromatin Buffer, and Heterochromatin Buffer.

### Sample Analysis

Nuclei isolated and/or manipulated via the use of lectins bound to a solid support may be analyzed via a variety of means. For example, they may be treated with different agents or under various biological or environmental conditions, and the effect on the nuclei examined. In certain embodiments, the effect on the amount or localization of various proteins within the nuclei is determined. Protein amounts and/or identity within nuclei or nuclear protein fractions may be analyzed via a variety of means known in the art, including but not limited to, mass spectrometry and/or immunological methods, such as western blotting and other methods using antibodies specific for nuclear proteins of interest.

In certain embodiments, after one or more of the cytoplasmic fraction, nuclear fraction, nucleoplasm fraction, euchromatin fraction, and/or heterochromatin fraction are obtained as described herein, they may be analyzed via a variety of means known in the art, e.g., to determine the identity of the proteins within the fraction(s). In particular embodiments, they are analyzed via mass spectrometry to determine the presence and/or absence of peptides and proteins present in the samples.

In certain embodiments, proteins in the sample(s) are digested using trypsin protease, followed by mass spectrometry analysis. Mass spectrometry data may be collected using a data-dependent acquisition strategy and peptides modified by the electrophilic label, e.g., NM-IAA, are identified based on the detection of the mass modification of NM-IAA (about 71.03711 Da) as a variable modification on cysteine residues using any proteomics search software.

In particular embodiments, samples may be analyzed using mass spectrometry (MS), such as tandem mass spectrometry (MS-MS), time-of-flight mass spectrometry (TOF-MS), quadrupole mass spectrometry (Q-MS), or any combination thereof. The samples may be analyzed using a combination of chromatographic and mass spectrometric techniques, such as gas chromatography-mass spectrometry (GC-MS), liquid chromatography-mass spectrometry (LC-MS), or high-pressure liquid chromatography-mass spectrometry (HPLC-MS).

### Applications

The use of lectins bound to solid surfaces provides the ability to process, manipulate, process, and/or analyze the nuclei for a variety of different applications. For example, nuclei bound to lectins bound to the solid surface may be treated to one or more different conditions, and then isolated via the solid surface, e.g., to examine biological effects of the one or more different conditions. In particular embodiments, having the nuclei bound to the solid support via the lectins allows for easy removal of liquids, e.g., containing various active molecules being tested, from the nuclei, e.g., by removing the liquid from the support-bound nuclei, or by removing the support-bound nuclei from the liquid. In particular embodiments, the nuclei may be subjected to a series of different conditions by moving the nuclei from one condition to another via the solid support to which they are bound. This may be done, e.g., using lectins bound to a magnetic bead, which can then be removed with associated nuclei using a magnet.

The methods disclosed herein may be used for a variety of purposes. For example, the methods disclosed herein may be used to compare nuclear proteomes across two or more conditions. For instance, the methods described herein may be used to characterize nuclear proteome changes in response to one or more external or internal perturbations. Such perturbations may include, but are not limited to, a change in cell state (e.g., cell cycle), cell environment, or exposure of the cell to a chemical treatment or physical stress. Such changes may be detected by changes in the location of proteins, e.g., proteins moving from one or more of the cytoplasm, nucleoplasm, euchromatin, or heterochromatin. For example, the methods may be used to identify functional systems or proteins, e.g., TFs, with alterations associated with an external or internal perturbations, such as alterations in cellular location.

In certain embodiments, the methods may be used to diagnose a disease or disorder, including any of those described herein, including but not limited to, cancer, infection, immunological disease, metabolic disease, cardiac disease, inflammatory disease, etc. For example, a cell sample obtained from a subject may be analyzed as described herein, and results related to the location of cellular proteins (e.g., transcription factors (TFs) or kinases) can be compared to the results obtained from healthy cells and/or diseased cells, or a predetermined set of results from healthy cells and/or diseased cells, thereby determining whether the cells are healthy or diseased. In certain embodiments, the methods are used to identify cytosolic and/or nuclear proteins, e.g., TFs, having a change in location in diseased cells as compared to healthy cells. In some embodiments, the methods are used to identify and/or analyze transcription factors, kinases, and/or proteins involved in nuclear import/trafficking.

In certain embodiments, the methods disclosed herein may be used to characterize small molecule degradation compounds. The methods described herein may be used to screen small molecule degraders in an unbiased manner to identify proteins targeted for degradation in response to treatment.

In certain embodiments, the methods disclosed herein may be used to assay cellular thermal shifts. The assay may detect compound engagement with the protein target in living cells by measuring changes in thermal stability of the protein. The methods described herein may be used to profile thermal stability of the nuclear proteome and to study compounds interacting with nuclear proteins.

In certain embodiments, the methods disclosed herein may be used to characterize genome edits. Genome editing with clustered regularly interspersed palindromic repeats (CRISPR)-based genome editing techniques, transcription activator-like effector nuclear (TALEN)-based genome editing techniques, zinc finger-based genome editing techniques, or other nuclease technologies can create mutations in the DNA of a cell. These mutations may result in global changes in the proteome and/or the nuclear proteome which may be detected with the methods described herein.

In certain embodiments, the methods disclosed herein may be used to enable single cell analysis, e.g., where cell counts are low, including but not limited to handling or manipulating nucleus from a single cell or from tens or hundreds of nuclei. Use of the methods disclosed herein to handle low numbers of nuclei is useful for single cell proteomics, genomics, and transcriptomics.

In certain embodiments, the methods disclosed herein may be used to isolate nuclei from tissues, including, e.g., previously fixed tissues, including but not limited to Formalin-fixed, Paraffin-Embedded (FFPE) tissues, paraformaldehyde (PFA) fixed tissues, or alcohol (e.g., ethanol or methanol) fixed tissues. Similarly, the methods mat be used to remove formalin, paraffin, or FDA from isolated nuclei. In some embodiments, the nuclei isolated from fixed tissues can be analyzed by flow cytometry or used for fluorescence-activated cell sorting (FACS).

### Definitions

Unless defined otherwise, all technical and scientific terms used herein have the same meaning as is commonly understood by one skilled in the art to which the claimed subject matter belongs. It is to be understood that the foregoing and the following descriptions are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise.

In this application, the use of “or” means “and/or” unless stated otherwise. Furthermore, use of the term “including” as well as other forms, such as “include”, “includes,” and “included,” is not limiting.

As used herein, ranges and amounts may be expressed as “about” a particular value or range. About also includes the exact amount. Hence “about 5 μL” means “about 5 μL” and also “5 μL.” Generally, the term “about” includes an amount that would be expected to be within experimental error. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “purify” means to remove a component from its natural environment or from one or more undesired components (e.g., non-nuclei cellular components), in part or completely.

As used herein, the term “separate” means to isolate two or more components from each other, in part or completely.

As used herein, the term “enrich” means to increase the concentration of a component, e.g., nuclei.

As used herein, the terms “purify,” “separate,” “enrich” and the like do not necessarily mean that the component is 100% removed from other components. For example, in certain embodiments, following purification, separation, or enrichment, the component may be present at an increased % concentration or molar concentration as compared to other components or at an increased ratio when compared to the component(s) from which it was separated. In certain embodiments, the concentration or ratio of the component is increase by at least 20%, at least 50%, at least 100%, at least two-fold, at least 5-fld, at least 10-fold, at least 20-fold, at least 50-fold, or at least 100-fold.

As used herein, the terms “lectin-coupled solid support” or “lectin-conjugated solid support” are used interchangeably and refer to a solid support, wherein one or more lectins is on the surface of the solid support. The one or more lectin can be attached to the solid support as described in the present disclosure or through any means known in the arts.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### EXAMPLES

### Example 1: Identification of Lectins that Bind to Glycans Enriched on the Nuclear Membrane

To determine glycans that are enriched on the nuclear membrane, 5 million MM1.S cells were subjected to chromatin extraction, and the cytosolic (containing the outer cell membrane) and insoluble (containing the nuclear membrane) fractions were analyzed using glycomic mass spectrometry. Gal indicates D-galactose; GlcNAc indicates N-acetylglucosamine; Man indicates D-mannose; Glc indicates D-glucose; and GalNAc indicates N-acetylgalactosamine. Paucimannose glycans include relatively simple mannose (Man) and N-acetylglucosamine (GlcNAc)-containing glycans.

TABLE 1
Lectin and glycan pairs that bind with specificity

| Lectin | Glycan epitope names |
|---|---|
| Amaranthus caudatus lectin | Galb1-3GalNAc, LacNAc |
| Datura Stramonium lectin | LacNAc, GlcNAcb1-4GlcNAc |
| Erythrina crista-galli lectin | LacNAc, galactose, GalNAc |
| Lycopersicon esculentum lectin | LacNAc |
| Maackia amurensis agglutinin I | 3′sulfo-LacNAc, 3′sulfo-Lac |
| Ricinus communis agglutinin I | LacNAc, galactose, GalNAc |

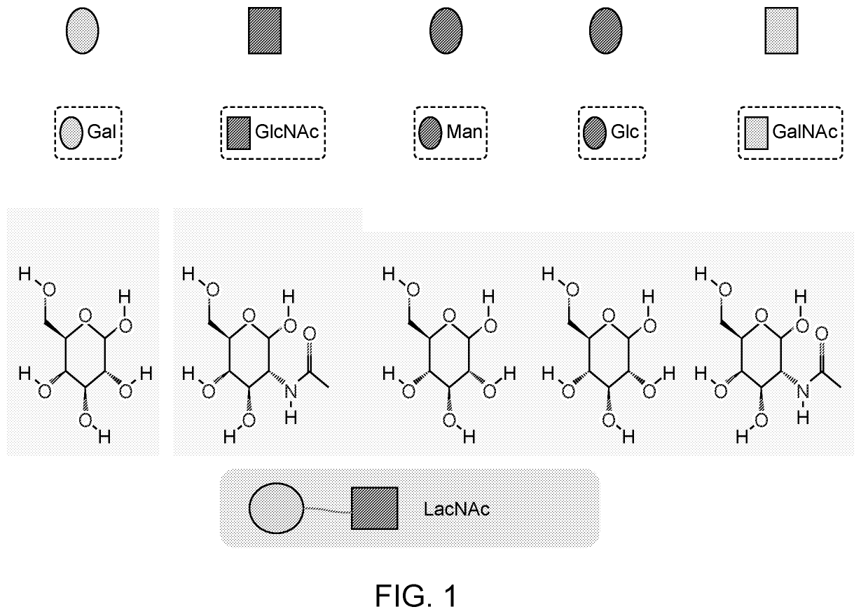

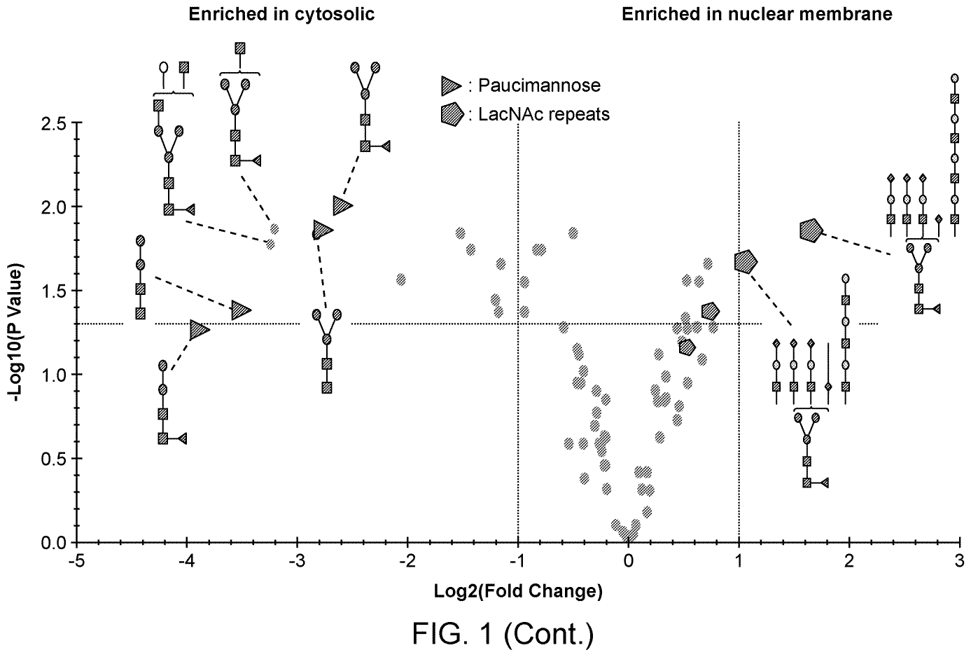

As shown in the volcano plot in FIG. 1, the cytosolic fraction was enriched with glycoproteins containing paucimannose glycans, whereas the insoluble fraction containing the nuclear membrane was enriched with glycoproteins containing LacNAcs (galactose and N-acetylglucosamine disaccharide). Lectins that specifically bind to these highly abundant nuclear membrane glycans were then identified by searching the National Cancer Institute's Center for Cancer Research Database of Anti-glycan Reagents (DAGR) for lectins that bind to the LacNAc epitope. These lectins include Amaranthus caudatus lectin, Datura stramonium lectin, Erythrina crista-galli lectin (ECL), Lycopersicon esculentum lectin, Maackia amurensis agglutinin I, and Ricinus communis agglutinin I (RCA), e.g., RCA120, which are shown in Table 1. Erythrina crista-galli lectin (ECL) and Ricinus communis agglutinin (RCA) preferentially bind to galactose, N-acetylgalactosamine (GalNAc), and N-acetyllactosamine (LacNAc) and were selected for use for handling and isolating cell nuclei to demonstrate the methods described herein.

### Example 2: Production of Lectin-Conjugated Beads

Magnetic beads were conjugated to lectins for use in isolation of cell nuclei as described herein.

TABLE 2
Formulations of buffers for preparing lectin-conjugated beads

| Buffer | Components |
|---|---|
| Bead wash buffer | 1x PBS |
| Bead conjugation buffer | 1X PBS containing 0.01% |
| | Tween-20 |
| Bead activation buffer | 20 mM HEPES, 10 mM KCl, |
| | 10 mM CaCl₂ 1 mM MnCl₂ |
| Nuclear extraction buffer | 20 mM HEPES, 10 mM KCl, |
| | 20% Glycerol, 1 mM MnCl_{2,} |
| | 0.1% Triton X-100 |
| EDTA-free Isotonic buffer | 10 mM Tris pH 8.0, 15 mM |
| | NaCl, 60 mM KCl |

Formulations of buffers used for the preparation of lectin-conjugated beads can be found in Table 2.

To prepare lectin-conjugated beads, 250 μl of magnetic streptavidin beads at a concentration of 4 mg/ml (1000 μg of magnetic beads) were pipetted into a 1.5 mL tube. The tube was then placed on a magnetic rack for 1 minute until the magnetic beads were collected to the side of the tube. The supernatant was carefully removed without disturbing the magnetic beads. To wash the beads, the tube was removed from the magnetic rack and the beads were resuspended in 500 μl of Bead wash buffer. The tube was then placed on a magnetic rack for 1 minute until the magnetic beads were collected to the side of the tube. The wash step was repeated two more times. After the three washes, the magnetic beads were resuspended into 250 μl (the original bead volume) of Bead conjugation buffer. 25 μl of biotinylated lectin suspension (such as biotinylated ECL or biotinylated RCA) at a concentration of 5 mg/mL was added to the tube. The tube was gently shaken (300-350 RPM) for 30 minutes at room temperature and placed on a magnetic rack for 1 minute until the magnetic beads were collected to the side of the tube. The beads were then washed as described previously and resuspended into 250 μl of Bead conjugation buffer and used for downstream applications or stored at 4° C. for future use.

### Example 3: Isolation of Cell Nuclei and Nuclear Fractions Using Lectin-Coated Magnetic Beads

Cell nuclei and nuclear fractions were prepared using lectin-coated magnetic beads.

TABLE 3
Formulations of buffers used for isolating cell nuclei

| Buffer | Components |
|---|---|
| Bead activation buffer | 20 mM HEPES, 10 mM KCl, |
| | 10 mM CaCl_{2,} 1 mM MnCl₂ |
| Nuclear extraction buffer | 20 mM HEPES, 10 mM KCl, |
| | 20% Glycerol, 1 mM MnCl_{2,} |
| | 0.1% Triton X-100 |
| EDTA-free isotonic buffer | 10 mM Tris pH 8.0, 15 mM |
| | NaCl, 60 mM KCl |
| Euchromatin buffer | 10 mM Tris pH 8.0, 250 mM |
| (low salt buffer) | NaCl, 1 mM EDTA pH 8.0 |
| Heterochromatin buffer | 10 mM Tris pH 8.0, 600 mM |
| (high salt buffer) | NaCl, 1 mM EDTA pH 8.0 |

Formulations of buffers used for the isolation of these cell components can be found in Table 3.

Lectin-conjugated magnetic beads were prepared as described in Example 2, and the beads were activated prior to their use. To activate the beads, 20 μL of beads per million cells was transferred to a 1.5 mL polypropylene tube. For example, 1200 μL of beads was transferred for a total of 12 samples that each contained 5 million cells. The tube containing the beads was placed on a magnetic tube rack and allowed to sit for 1 minute until the beads were collected to the side of the tube by the magnet. The supernatant was then removed and discarded. The tube was removed from the magnet and washed with Bead Activation Buffer three times. For each wash, the beads were fully resuspended into 1200 μL of Bead Activation Buffer. Then, the tube containing the beads was placed on a magnetic tube rack and allowed to sit for 1 minute until the beads were collected to the side of the tube by the magnet. The supernatant containing the Bead Activation Buffer was removed and discarded. After the three washes, the lectin-conjugated beads were resuspended into 1200 μL of Bead Activation Buffer and ready for use.

As illustrated in FIG. 2, to isolate cell nuclei and nuclear fractions using the activated lectin-coated magnetic beads, cells were collected into 1.5 mL polypropylene tubes and centrifuged at 400 RCF for 5 minutes at 4° C. The supernatant was removed and discarded. The cell pellets were resuspended in 1 mL of ice cold 1×PBS then centrifuged at 400 RCF for 5 minutes at 4° C. The supernatant was removed and discarded. Each pellet was resuspended in 200 μL of Nuclear Extraction Buffer (NEB) and incubated for 10 minutes at 4° C. After the incubation, the samples containing the isolated nuclei were centrifuged at 400 RCF for 5 minutes at 4° C. The supernatant was removed and retained as the Cytosolic Fraction.

The pellet containing the nuclei was resuspended in 200 μL of EDTA-free Isotonic Buffer. Activated lectin-conjugated magnetic beads were added to each sample at a volume of 20 μL per million cells. In this example, 100 μL of activated beads was added to each sample, which contained 5 million cells. The activated beads were mixed thoroughly with the pellet and the Isotonic Buffer by pipetting up and down. The samples were then incubated at 4° C. with gentle shaking for 20 minutes to allow for the cell nuclei to bind to the lectin-conjugated magnetic beads. The tubes containing the nuclei and beads suspension were placed onto a magnetic rack and allowed to sit for at least one minute for the beads to pellet and collect onto the side of the tube. The supernatant was carefully removed without disturbing the pellet and retained as the Nucleoplasm Fraction.

The tubes were then removed from the magnetic rack, and the pellets were resuspended in 200 μL of euchromatin buffer and incubated at 4° C. for 15 minutes. The tubes were then returned to the magnetic rack and allowed to sit for 1 minute for the beads to pellet and collect onto the side of the tube. The supernatant was carefully removed and retained as the Euchromatin Fraction.

The tubes were then removed from the magnetic rack, and the pellets were resuspended in 200 μL of heterochromatin buffer and incubated at 4° C. for 15 minutes. The tubes were then returned to the magnetic rack and allowed to sit for 1 minute for the beads to pellet and collect onto the side of the tube. The supernatant was carefully removed and retained as the Heterochromatin Fraction. The remaining pellet containing the insoluble fraction was stored for future use. The insoluble fraction contains cell debris, including chromatin and insoluble chromatin-associated proteins, and can be further analyzed, e.g., for histone post-translational modifications, or for isolating nucleic acids for further analysis.

### Example 4: Use of Erythrina crista-Galli Lectin (ECL) and Ricinus communis Agglutinin (RCA) for Nuclear Protein Extraction

To compare the use of ECL or RCA-conjugated magnetic beads with ConA-conjugated magnetic beads for cell nuclei isolation, the nucleoplasm, euchromatin, and heterochromatin fractions were extracted from 4 million MM1.S cells, as described in Example 3 above.

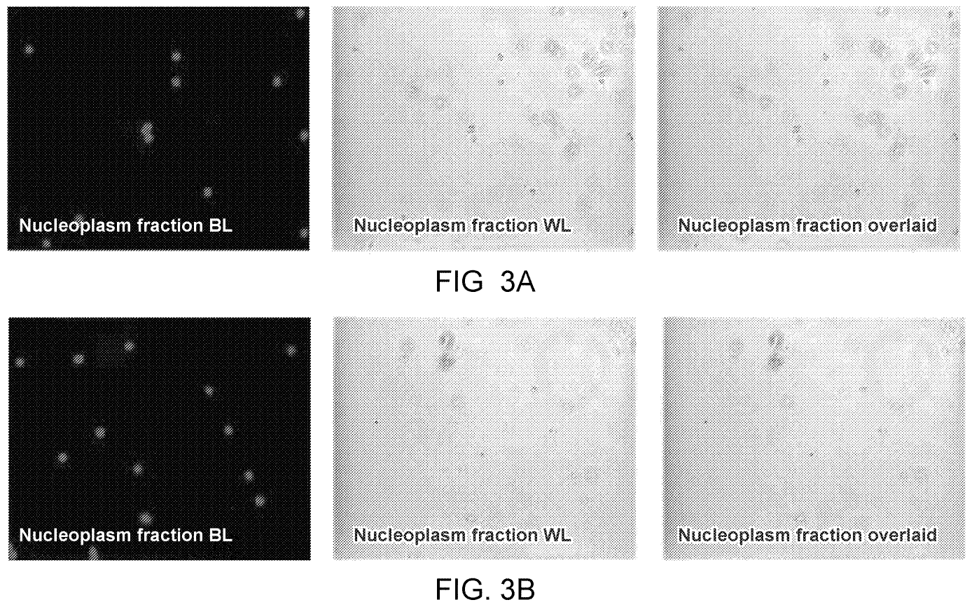

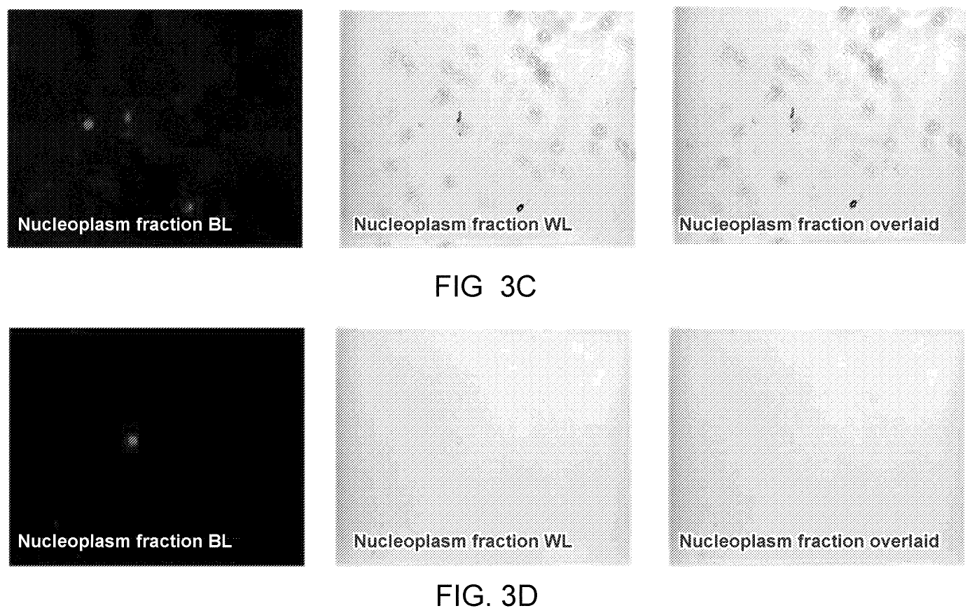

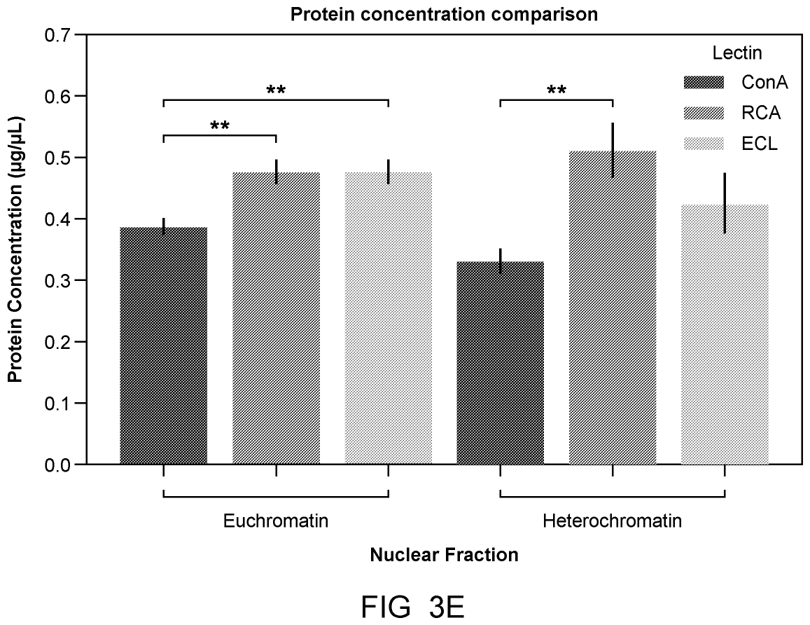

Images of the nucleoplasm fractions were taken using an EVOS FLoid imaging system. As shown in the blue light images in FIGS. 3A-3D, the nucleoplasm fractions extracted using ConA-conjugated magnetic beads (FIGS. 3A-3B) have more left-behind nuclei when compared to that of RCA-conjugated magnetic beads (FIG. 3C) and ECL-conjugated magnetic beads (FIG. 3D). Additionally, the brightfield images in FIGS. 3A-3D show fewer nuclei left behind with the RCA and ECL-conjugated magnetic beads, which demonstrates these lectins are more effective at binding to and moving nuclei than ConA.

The amount of protein isolated in each of the euchromatin and heterochromatin fractions using the ConA, RCA, and ECL-conjugated magnetic beads was determined using a 660-nm Protein Assay. As shown in FIG. 3E, both the euchromatin fractions extracted by RCA and ECL and the heterochromatin fractions extracted using RCA-conjugated magnetic beads yielded significantly higher amounts of protein compared to that of ConA. These results show that ECL and RCA are more efficient than ConA at nuclear protein extraction. (p-values of <0.01 denoted by “**”).

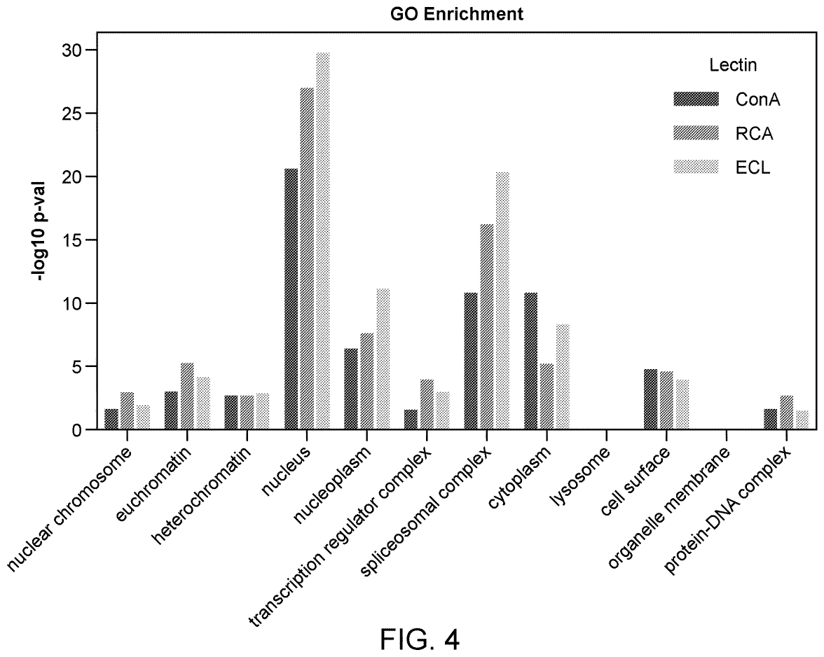

The combined chromatin (combining equal volumes of euchromatin and heterochromatin fraction) isolated using ConA, ECL, or RCA-conjugated magnetic beads were analyzed by LC-MS, and GO (gene ontology) enrichment analysis was used to evaluate the protein content of each sample. As shown in FIG. 4, RCA- and ECL-conjugated beads successfully enriched nuclear proteins, with enrichment of proteins associated with the nuclear chromosome, euchromatin, heterochromatin, nucleus, nucleoplasm, and protein-DNA complex. Additionally, RCA and ECL-conjugated magnetic beads led to higher enrichment of proteins associated with the nucleus compared to that of ConA-conjugated magnetic beads. Importantly, use of ConA resulted in higher enrichment for cytoplasmic and cell-surface proteins compared to ECL and RCA, demonstrating that ECL and RCA are superior lectins for use in isolating cell nuclei for protein extraction.

### Example 5: Automated Nuclear Protein Extraction Using Lectin-Coated Magnetic Beads in a 96-Well Format

To test automation and applicability to drug discovery for high-throughput nuclear protein extraction and fractionation using ECL-conjugated magnetic beads, a Thermo Scientific KingFisher™ Apex System was used to extract the nucleoplasm, chromatin, and insoluble fractions from treated THP-1 cells.

In the present example, THP-1 cells were collected and seeded in a V-bottom 96-well plate with wells containing the drugs dBET6, VTP56345345, or DMSO. Cells in 170 μL of media (RPMI-1 containing 10% fetal bovine serum and 1× penicillin/streptomycin) were transferred into each well of the plate. The cells were incubated for 4 hours at 37° C. After the treatment incubation, the cells were transferred to a 200 μL KingFisher™ plate and then centrifuged at 400 RCF for 5 minutes at 4° C. The supernatant was removed and discarded. 170 μL of ice-cold 1×PBS was added to each well of the treated cells and then centrifuged at 400 RCF for 5 minutes at 4° C.

The supernatant was removed and discarded. 140 μL of NEB was added to each well and the cells were pipetted up and down at least three times to mix. 10 μL of ECL-conjugated and activated (as described in Example 4 above) beads were added to each well. The cell and bead suspension was pipetted up and down to mix. The plate was covered with a clear plastic seal and incubated with gentle shaking (450 RPM) on a Thermomixer for 20 minutes at 4° C.

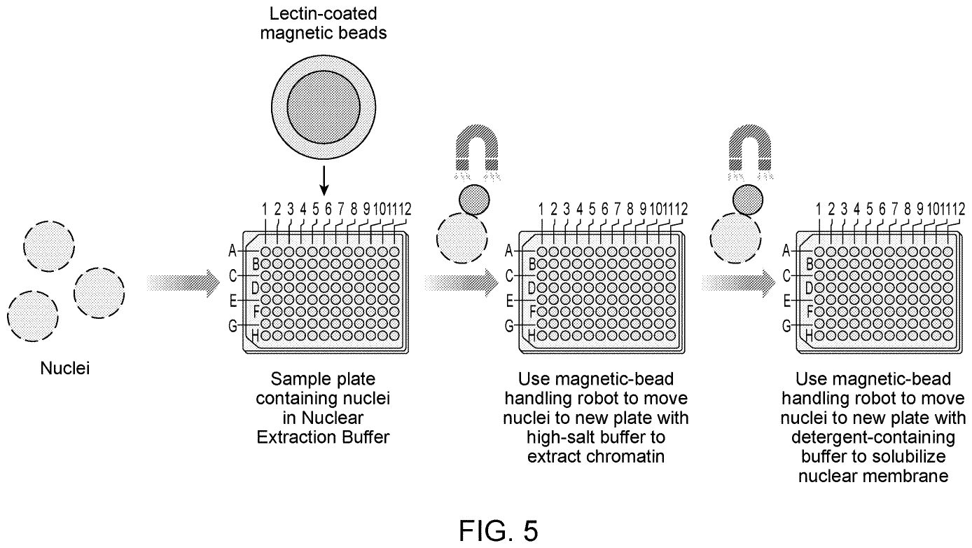

After incubation, the automated chromatin extraction by salt separation (ChESS) program was then initiated, which directed the KingFisher™ system to rotate through the loaded plates in sequential order, as shown in FIG. 5. The 96-well plate containing the samples was loaded into the KingFisher™ system, along with three other plates. The first 96 deep-well plate contained 125 μL per well of isotonic buffer (10 mM Tris pH 8.0, 15 mM NaCl, 60 mM KCl) to extract nucleoplasm and the second contained 125 μL per well of heterochromatin extraction buffer (10 mM Tris pH 8.0, 600 mM NaCl, 1 mM EDTA pH 8.0) to extract chromatin. Lastly, the third plate, a KingFisher™ 200 μl plate, contained 125 μL per well of insoluble buffer (1% sodium dodecyl sulfate, 10 mM MgCl2, in phosphate-buffered saline). KingFisher™ 96 tip combs (Thermo Fisher Scientific; Cat #97002534) for deep-well magnets were assembled with the 96 deep-well plates as directed by manufacturer instructions and used for sample transfer. The three plates containing isolated nuclear fractions (nucleoplasm, chromatin, or insoluble) were retained for downstream processing at the end of the KingFisher™ program run.

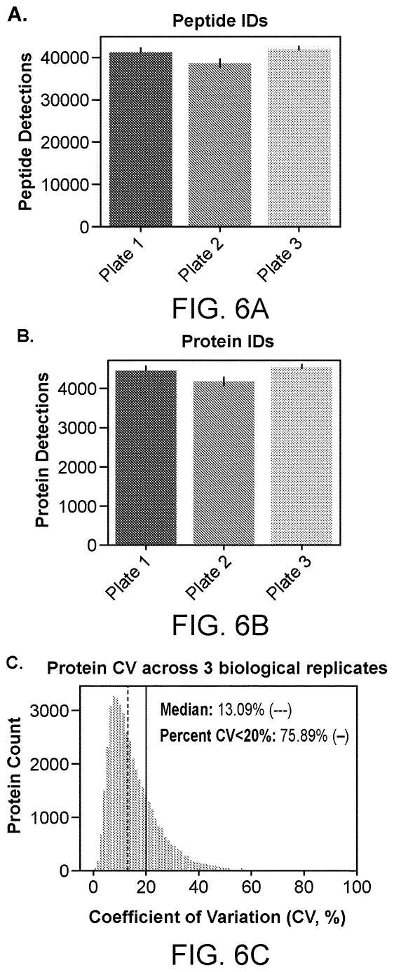

The above process was repeated two additional times for a total of three biological replicates. The chromatin fractions of these plates were digested with trypsin and analyzed by LC-MS using a Bruker timsTOF Ultra. As shown in FIGS. 6A-6B, high peptide and protein detections were observed across conditions and across the plate replicates, and the median coefficient of variation (CV) was calculated to be less than 13.09% for all proteins across plates, and the total percentage of proteins with a CV of less than 20% was 75.89% (FIG. 6C). Together, these results demonstrate the protocol's robustness in varying sample environments and between replicates.

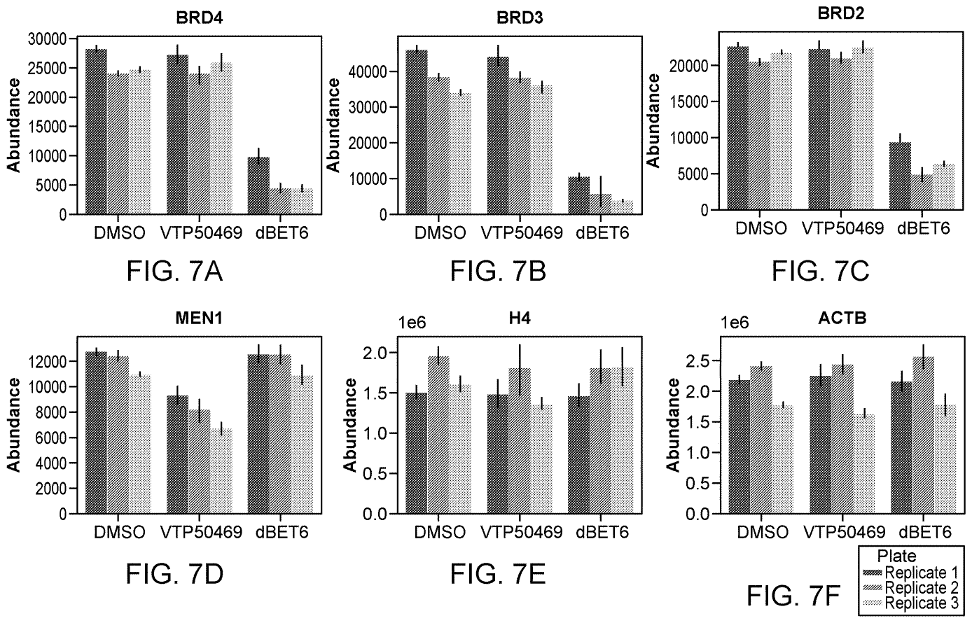

In addition, FIGS. 7A-7C show that samples treated with the compound dBET6 have a decreased expression of the BRD family proteins, which are the validated targets of that compound. FIG. 7D shows that samples treated with the compound VTP50469 have a decreased level of MEN1 expression, which is the validated target protein of that compound. Importantly, control proteins such as H4 and ACTB demonstrate stable expression, remaining largely unchanged between treatment conditions (FIGS. 7E-7F). These results in FIGS. 7A-7F illustrate that this protocol is capable of characterizing the accurate biological impact of a compound on the protein content of the nucleus, and these effects are captured across replicates with low technical variation. Thus, these figures demonstrate that automated nuclear extraction of proteins using lectin coated magnetic beads can be performed reproducibly in a 96-well format, and be applied to high-throughput proteomics for drug discovery applications or other applications requiring large-scale processing of the nuclear and chromatin-associated proteome.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

### REFERENCES

(1) Monneron, A.; Segretain, D. Extensive Binding of Concanavalin A to the Nuclear Membrane. FEBS Lett. 1974, 42 (2), 209-213. https://doi.org/10.1016/0014-5793 (74) 80787-8.
(2) Stanley, P.; Moremen, K. W.; Lewis, N. E.; Taniguchi, N.; Aebi, M. N-Glycans; Cold Spring Harbor Laboratory Press, 2022. https://doi.org/10.1101/glycobiology.4e.9.

We/I claim:

1. A method of associating cell nuclei to a solid support, comprising contacting nuclei in a sample with a solid support comprising one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc), thereby associating the nuclei to the solid support.

2. The method of claim 1, wherein the one or more lectin comprises Erythrina crista-galli lectin (ECL), Ricinus communis agglutinin (RCA), Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin.

3. The method of claim 1 or claim 2, wherein the solid support is a bead.

4. The method of claim 3, wherein the bead is a magnetic bead.

5. The method of any one of claims 1-4, for use in isolating or extracting the nuclei from cells, cell lysate, and/or other non-nuclear cellular components.

6. The method of claim 5, wherein the nuclei are contacted with the solid support while in a sample comprising cells, cell lysate, and/or other non-nuclear cellular components.

7. The method of claim 6, further comprising separating or removing the solid support and associated nuclei from the sample.

8. The method of claim 7, wherein the solid support and associated nuclei are separated or removed using a magnet.

9. The method of any one of claims 1-4, for use in transferring, moving, or washing the cell nuclei.

10. The method of claim 9, wherein the method further comprises separating or removing the solid support and associated nuclei from the sample, and optionally washing the nuclei.

11. The method of claim 10, wherein the solid support and associated nuclei are removed using a magnet.

12. The method of any one of claims 1-4, for use in immobilizing the cell nuclei.

13. The method of any one of claims 1-4, for use in enriching the cell nuclei.

14. A method of extracting nuclear proteins from cell nuclei, the method comprising:
(a) contacting nuclei with a first buffer to extract proteins from the nuclei;
(b) contacting the nuclei with a solid surface comprising one or more lectin coupled to a surface of the solid support, wherein the one or more lectin binds to N-acetyllactosamine (LacNAc), galactose, N-acetylgalactosamine (GalNAc), and/or N-acetylglucosamine (GlcNAc), thereby binding the nuclei to the solid support; and
(c) separating the buffer and the nuclei bound to the solid support, wherein the buffer comprises the nuclear proteins.

15. The method of claim 14, wherein the buffer is used to extract nucleoplasm proteins, chromatin-associated proteins, euchromatin-associated proteins, and/or heterochromatin-associated proteins.

16. The method of claim 14 or claim 15, wherein the buffer is an isotonic buffer to extract nucleoplasm proteins.

17. The method of claim 14 or claim 15, wherein the buffer is a low salt buffer to extract euchromatin-associated proteins.

18. The method of claim 14 or claim 15, wherein the buffer is a high salt buffer to extract heterochromatin-associated proteins.

19. The method of claims 14-18, wherein the solid support comprises beads.

20. The method of claim 19, wherein the beads are magnetic beads.

21. The method of claim 20, wherein the method comprises using a magnet to separate the solid support and associated nuclei from the buffer prior to step (c).

22. The method of any one of claims 14-21, wherein the method comprises repeating steps (a)-(c) using a second buffer.

23. The method of claim 22, wherein the method comprises repeating steps (a)-(c) using a third buffer.

24. The method of claim 23, wherein the first buffer is an isotonic buffer to extract nucleoplasm proteins, the second buffer is a low salt buffer to extract euchromatin proteins, and the third buffer is a high salt buffer to extract heterochromatin proteins.

25. The method of any one of claims 14-23, wherein the method is a high-throughput method, wherein the nuclei are present in a first buffer within multiple wells of a sample plate, the solid support is a magnetic bead, and nuclei bound to the solid support are removed from the multiple wells using magnets.

26. The method of claim 25, wherein following removal from the multiple wells comprising the first buffer, the nuclei are placed into a second buffer within multiple wells of a sample plate, and the nuclei bound to the solid support are then removed from the multiple wells using magnets.

27. The method of claim 26, wherein following removal from the multiple wells comprising the second buffer, the nuclei are placed into a third buffer within multiple wells of a sample plate, and the nuclei bound to the solid support are then removed from the multiple wells using magnets.

28. The method of claim 27, wherein the first buffer is an isotonic buffer to extract nucleoplasm proteins, the second buffer is a low salt buffer to extract euchromatin proteins, and the third buffer is a high salt buffer to extract heterochromatin proteins.

29. The method of any one of claims 14-23, wherein the method is a high-throughput method, wherein the nuclei are present in a first buffer within multiple wells of a sample plate, the solid support is a magnetic bead, and nuclei bound to the solid support are separated from the buffer using magnets and the buffer is transferred or removed.

30. The method of claim 29, wherein following removal of the first buffer from the multiple wells, a second buffer is added to the nuclei bound to the solid support within the multiple wells of a sample plate, and the nuclei bound to the solid support are separated from the buffer using magnets and the second buffer is transferred or removed.

31. The method of claim 30, wherein following removal of the second buffer from the multiple wells, a third buffer is added to the nuclei bound to the solid support within the multiple wells of a sample plate, and the nuclei bound to the solid support are separated from the buffer using magnets and the third buffer is transferred or removed.

32. The method of claim 31, wherein the first buffer is an isotonic buffer to extract nucleoplasm proteins, the second buffer is a low salt buffer to extract euchromatin proteins, and the third buffer is a high salt buffer to extract heterochromatin proteins.

33. A solid support, wherein one or more lectin that binds to N-acetyllactosamine (LacNAc), galactose, N-acetylgalactosamine (GalNAc), and/or N-acetylglucosamine (GlcNAc) is coupled to a surface of the solid support.

34. The solid support of claim 33, wherein the one or more lectin comprises Erythrina crista-galli lectin (ECL), Ricinus communis agglutinin (RCA), Amaranthus caudatus lectin, Datura stramonium lectin, Lycopersicon esculentum lectin, Maackia amurensis agglutinin I, Solanum tuberosum lectin, soybean agglutinin, Vicia villosa lectin, and/or Wisteria floribunda lectin.

35. The solid support of claim 34, wherein the one or more lectin comprises ECL.

36. The solid support of claim 34, wherein the one or more lectin comprises RCA, optionally wherein the RCA is RCA120 or RCAI.

37. The solid support of any one of claims 33-36, wherein the solid support is a bead.

38. The solid support of claim 37, wherein the bead is a magnetic bead.

39. The solid support of any one of claims 33-38, wherein the one or more lectins are coupled to the surface of the solid support via a biotin-streptavidin conjugation.

40. A kit comprising:
(a) the solid support of any one of claims 33-39; and
(b) one or more buffers suitable for extracting proteins from mammalian cell nuclei.

41. The kit of claim 40, further comprising one or more buffers suitable for lysing mammalian cells.

42. The kit of claim 40 or claim 41, comprising one or more buffers selected from:
an isotonic buffer, a low salt buffer, and a high salt buffer.

43. A system comprising:
(a) the solid support of any one of claims 33-39, wherein the solid support is a magnetic bead;
(b) one or more buffers suitable for extracting proteins from mammalian cell nuclei; and
(c) one or more magnets.

44. The system of claim 43, further comprising one or more buffers suitable for lysing cells.

45. The system of claim 43 or claim 44, comprising one or more buffers selected from: an isotonic buffer, a low salt buffer, and a high salt buffer.

46. The system of any one of claims 43-45, where the one or more magnets are present in a magnetic bead handling robot or liquid handling robot.

47. The system of any one of claims 43-46, wherein the system comprises a multi-well plate, and wherein the one or more magnets are configured to fit into the wells of the multi-well plate or configured to sit below the multi-well plate to pull the magnetic beads to the bottom or side of the well.
